(19) United States
(12) Patent Application Publication
(10) Pub. No.: US 2025/0040858 A1
(43) Pub. Date: Feb. 6, 2025

(54) AF BURDEN ENHANCEMENT

(71) Applicant: Cardiac Pacemakers, Inc., St. Paul, MN (US)

(72) Inventors: David L. Perschbacher, Blaine, MN (US); Deepa Mahajan, North Oaks, MN (US); Sunipa Saha, Shoreview, MN (US)

(21) Appl. No.: 18/779,706

(22) Filed: Jul. 22, 2024

Related U.S. Application Data
(60) Provisional application No. 63/529,973, filed on Jul. 31, 2023.

(51) Int. Cl. A61B 5/361; A61B 5/00; A61B 5/333

(52) U.S. Cl. CPC A61B 5/361; A61B 5/0006; A61B 5/333

(57) ABSTRACT

Systems and methods are disclosed to determine and record one or more key metrics of atrial fibrillation for a patient, including determining indications of atrial fibrillation of the patient in respective detection windows of a day using received physiologic information, recording first physiologic information of the patient at a first sampling frequency for the determined indications of atrial fibrillation of the patient up to and not exceeding a first threshold of the medical device system for transmission to a remote device, and determining and recording one or more key metrics of atrial fibrillation for the determined indications of atrial fibrillation of the patient at a second sampling frequency lower than the first sampling frequency without regard to the first threshold.

### CLAIM OF PRIORITY

This application claims the benefit of U.S. Provisional Application No. 63/529,973 filed on Jul. 31, 2023, which is hereby incorporated by reference in its entirety.

### TECHNICAL FIELD

This document relates generally to medical devices and more particularly to systems and methods to determine an atrial fibrillation burden for a patient.

### BACKGROUND

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

An arrhythmia is an abnormal heart rhythm (e.g., fast, slow, irregular, etc.). Arrhythmias include, among others, bradycardia, tachycardia, premature, extra, or skipped heart beats, and atrial or ventricular fibrillation affecting one or more chambers of the heart. Atrial fibrillation (AF) is as an abnormal heart rhythm characterized by rapid and irregular activity in the left or right atria of the heart. Atrial fibrillation is commonly associated with a reduction in cardiac output, an increased risk of heart failure, dementia, and stroke. Risk factors for atrial fibrillation include, among others, high blood pressure, heart failure, valvular heart disease, chronic obstructive pulmonary disorder (COPD), obesity, and sleep apnea.

Ambulatory medical devices (AMDs), including implantable, subcutaneous, wearable, or one or more other medical devices, etc., can monitor, detect, or treat various conditions, including heart failure, atrial fibrillation, etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

### SUMMARY

Systems and methods are disclosed to determine and record one or more key metrics of atrial fibrillation for a patient, including determining indications of atrial fibrillation of the patient in respective detection windows of a day using received physiologic information, recording first physiologic information of the patient at a first sampling frequency for the determined indications of atrial fibrillation of the patient up to and not exceeding a first threshold of the medical device system for transmission to a remote device, and determining and recording one or more key metrics of atrial fibrillation for the determined indications of atrial fibrillation of the patient at a second sampling frequency lower than the first sampling frequency without regard to the first threshold. In certain examples, an indication of atrial fibrillation burden of the patient can be determined using the one or more key metrics of atrial fibrillation, such as using a comparison of the one or more key metrics to commensurate metrics for adjudicated episodes. In other examples, one or more modes or operations of one or more components of a medical device system can be transitioned, altered, or controlled using the one or more key metrics of atrial fibrillation.

An example of subject matter (e.g., a medical device system) may comprise a signal receiver circuit configured to receive physiologic information of a patient; and an assessment circuit configured to: determine indications of atrial fibrillation of the patient in respective detection windows of a day using the received physiologic information; record first physiologic information of the patient at a first sampling frequency for the determined indications of atrial fibrillation of the patient up to and not exceeding a first threshold of the medical device system for transmission to a remote device; and determine and record one or more key metrics of atrial fibrillation for the determined indications of atrial fibrillation of the patient at a second sampling frequency lower than the first sampling frequency without regard to the first threshold.

In an example, which may be combined with any one or more examples described herein, the assessment circuit may be configured to determine and record one or more key metrics of atrial fibrillation for the determined indications of atrial fibrillation without regard to a limit, optionally including to determine and record one or more key metrics of atrial fibrillation for the determined indications of atrial fibrillation up to and exceeding the first threshold.

In an example, which may be combined with any one or more examples described herein, the first threshold may comprise or be representative of a first data limit for storage or transmission by the assessment circuit for a determined indication of atrial fibrillation, a time period, or a period between sequential transmissions to the remote device.

In an example, which may be combined with any one or more examples described herein, the first threshold may comprise a daily threshold for determined indications of atrial fibrillation.

In an example, which may be combined with any one or more examples described herein, the assessment circuit may be configured to cease recording the first physiologic information of the patient for a remainder of the day after an amount of the recorded first physiologic information for the day has met or exceeded the first threshold.

In an example, which may be combined with any one or more examples described herein, the assessment circuit may be configured to determine and record the one or more key metrics of atrial fibrillation for one or more detection windows of each of the determined indications of atrial fibrillation of the patient for transmission to the remote device.

In an example, which may be combined with any one or more examples described herein, the assessment circuit may be configured to determine and record the one or more key metrics of atrial fibrillation for each detection window of the determined indications of atrial fibrillation.

In an example, which may be combined with any one or more examples described herein, the assessment circuit may be configured to determine and record the one or more key metrics of atrial fibrillation for each detection window of the determined indications of atrial fibrillation and at least one detection window preceding or following the determined indications of atrial fibrillation.

In an example, which may be combined with any one or more examples described herein, each determined indication of atrial fibrillation may comprise one or more detection windows.

In an example, which may be combined with any one or more examples described herein, the assessment circuit may be configured to determine and record the one or more key metrics of atrial fibrillation for each detection window of the day, including detection windows with and without determined indications of atrial fibrillation.

In an example, which may be combined with any one or more examples described herein, the subject matter may optionally comprise an implantable or an ambulatory medical device including the signal receiver circuit and the assessment circuit.

In an example, which may be combined with any one or more examples described herein, the detection window may have a duration between 30 seconds and 2 minutes.

In an example, which may be combined with any one or more examples described herein, the first physiologic information of the patient at the first sampling frequency may comprise an ECG signal of the patient at a sampling frequency greater than 100 samples per second for the determine indications of atrial fibrillation up to and not exceeding the first threshold of the medical device system,

In an example, which may be combined with any one or more examples described herein, the one or more key metrics of atrial fibrillation may comprise one or more measures or values representative of one or more detection windows of the determined indications at the second sampling frequency having fewer than 5 samples per detection window.

In an example, which may be combined with any one or more examples described herein, the one or more key metrics of atrial fibrillation may comprise, for the one or more detection windows of each determined indication of atrial fibrillation, a measure or value of one or more of: R-R variability; timing intervals between successive valid R waves; R-wave morphology; P-wave presence; and heart rate.

In an example, which may be combined with any one or more examples described herein, the measure or value of one or more of R-R variability, timing intervals between successive valid R waves, and heart rate comprises an indication that one or more of R-R variability, timing intervals between successive valid R waves, and heart rate is above or below a patient-specific or population threshold for the one or more detection windows of each determined indication of atrial fibrillation.

In an example, which may be combined with any one or more examples described herein, the assessment circuit may be configured to determine an indication of atrial fibrillation burden for the patient using the one or more recorded key metrics of atrial fibrillation.

In an example, which may be combined with any one or more examples described herein, the assessment circuit may be configured to determine the indication of atrial fibrillation burden for the patient using a comparison of the one or more recorded key metrics to commensurate metrics for adjudicated episodes.

An example of subject matter (e.g., a method, such as of operating a medical device system) may comprise receiving physiologic information of a patient using a signal receiver circuit and, using an assessment circuit: determining indications of atrial fibrillation of the patient in respective detection windows of a day using the received physiologic information; recording first physiologic information of the patient at a first sampling frequency for the determined indications of atrial fibrillation of the patient up to and not exceeding a first threshold for transmission to a remote device; and determining and recording one or more key metrics of atrial fibrillation for the determined indications of atrial fibrillation of the patient at a second sampling frequency lower than the first sampling frequency without regard to the first threshold.

In an example, which may be combined with any one or more examples described herein, determining and recording the one or more key metrics of atrial fibrillation for the determined indications of atrial fibrillation of the patient without regard to the first threshold may optionally comprise determining and recording one or more key metrics of atrial fibrillation for the determined indications of atrial fibrillation without regard to a limit, optionally including determining and recording one or more key metrics of atrial fibrillation for the determined indications of atrial fibrillation up to and exceeding the first threshold.

In an example, which may be combined with any one or more examples described herein, the first threshold may optionally comprise a daily threshold for determined indications of atrial fibrillation.

In an example, which may be combined with any one or more examples described herein, the subject matter may optionally comprise ceasing recording the first physiologic information of the patient for a remainder of the day after an amount of the recorded first physiologic information for the day has met or exceeded the first threshold.

In an example, which may be combined with any one or more examples described herein, determining and recording the one or more key metrics of atrial fibrillation may optionally comprise determining and recording the one or more key metrics of atrial fibrillation for each detection window of the day, optionally including detection windows with and without determined indications of atrial fibrillation.

In an example, which may be combined with any one or more examples described herein, determining and recording the one or more key metrics of atrial fibrillation may optionally comprise determining and recording the one or more key metrics of atrial fibrillation for one or more detection windows of each of the determined indications of atrial fibrillation of the patient for transmission to the remote device.

In an example, which may be combined with any one or more examples described herein, each determined indication of atrial fibrillation may optionally comprise one or more detection windows.

In an example, which may be combined with any one or more examples described herein, the detection window may optionally have a duration between 30 seconds and 2 minutes.

In an example, which may be combined with any one or more examples described herein, the first physiologic information of the patient at the first sampling frequency may optionally comprise an ECG signal of the patient at a sampling frequency greater than 100 samples per second for the determine indications of atrial fibrillation up to and not exceeding the first threshold of the medical device system.

In an example, which may be combined with any one or more examples described herein, the one or more key metrics of atrial fibrillation may optionally comprise one or more measures or values representative of one or more detection windows of the determined indications at the second sampling frequency having fewer than 5 samples per detection window.

In an example, which may be combined with any one or more examples described herein, the one or more key metrics of atrial fibrillation may optionally comprise, for the one or more detection windows of each determined indication of atrial fibrillation, a measure or value of one or more of: R-R variability timing intervals between successive valid R waves; R-wave morphology; P-wave presence, and heart rate.

In an example, which may be combined with any one or more examples described herein, the subject matter may optionally comprise determining an indication of atrial fibrillation burden for the patient using a comparison of the one or more recorded key metrics to commensurate metrics for adjudicated episodes.

In an example, a system or apparatus may optionally combine any portion or combination of any portion of any one or more of the examples above, may optionally combine any portion or combination of any portion of any one or more of the examples above to comprise “means for” performing any portion of any one or more of the functions or methods of the examples above, or at least one “non-transitory machine-readable medium” including instructions that, when performed by a machine, cause the machine to perform any portion of any one or more of the functions or methods of the examples above.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example diagram of daily atrial fibrillation burden.

FIG. 2 illustrates an example method for determining an indication of atrial fibrillation and recording ECG information associated with such determined indication of atrial fibrillation with existing hardware limitations.

FIG. 3 illustrates an example method for determining and recording one or more key metrics of atrial fibrillation or atrial fibrillation burden over a period of time without substantially exceeding or impacting existing hardware limitations.

FIG. 4 illustrates an example medical device system.

FIG. 5 illustrates an example patient management system.

FIG. 6 illustrates an example machine upon which any one or more of the techniques discussed herein may perform.

### DETAILED DESCRIPTION

Implantable or ambulatory medical devices can include, or be configured to receive cardiac electrical information from, one or more electrodes located within, on, or proximate to a heart, such as coupled to a lead and located in one or more chambers of the heart or within the vasculature of the heart near one or more chambers. Implantable or ambulatory medical devices can additionally include or be configured to receive mechanical acceleration information from one or more accelerometer sensors to determine and monitor patient acceleration information, such as cardiac acceleration or vibration information associated with blood flow or movement in the heart or patient vasculature (e.g., heart sounds, cardiac wall motion, etc.), patient physical activity or position information (e.g., patient posture, activity, steps, etc.), respiration information (e.g., respiration rate, volume, phase, breathing sounds, etc.), impedance information, plethysmograph information, chemical information, temperature information, or other physiologic information of the patient.

Arrhythmia events, including potential arrhythmia events, such as atrial fibrillation events or potential events, can be detected using sensed or received cardiac electrical information, including, for example, detected atrial or ventricular events (e.g., beats, r-waves, p-waves, etc.) or intervals therebetween occurring within a detection window, often between 30 seconds and 2 minutes, though in certain examples longer or shorter, such as between 15 seconds and 5 minutes, etc. Ambulatory medical devices can determine, for example, using timing information between events, in certain examples, in combination with one or more other detected events, whether atrial fibrillation is present or not in each detection window, and can additionally determine to store or transmit sensed or detected information, such as for transmission to a remote device, based on the determination. In certain examples, the ambulatory medical device can aggregate information from multiple sensors, detect various events using information from each sensor separately or in combination, update a detection status based on the information, and transmit a message or an alert to one or more remote devices that a detection has been made, that information has been stored or transmitted, such that one or more additional processes or systems can use the stored or transmitted detection or information for one or more other review or processes.

Atrial fibrillation detection algorithms commonly rely on cardiac electrical information features, such as cardiac intervals between successive R waves, individual beat-to-beat rate variance, etc. Examples of atrial fibrillation detection algorithms, including various atrial fibrillation detection parameters and criteria, can be found, for example, in the commonly assigned Krueger et al. U.S. patent application Ser. No. 14/825,669, titled “Atrial Fibrillation Detection Using Ventricular Rate Variability” (herein, “the '669 application”); Perschbacher et al. U.S. patent application Ser. No. 15/082,440, titled “Atrial Fibrillation Detection” (herein, “the '440 application”); Krueger et al. U.S. patent application Ser. No. 15/341,565, titled “Method and Apparatus for Enhancing Ventricular Based Atrial Fibrillation Detection Using Atrial Activity” (herein, “the '565 application”); and Perschbacher et al. U.S. patent application Ser. No. 15/864,953, titled “Atrial Fibrillation Discrimination Using Heart Rate Clustering” (herein, “the '953 application”), each of which are hereby incorporated by reference in their entireties, including their disclosure of atrial fibrillation detection and atrial fibrillation detection algorithms including, for example: atrial fibrillation detection using pairs of ventricular information detected from a ventricle, including rate changes and rate change characteristics, and determination of valid heart beats or intervals using various characteristics, including threshold rates, intervals, morphology criterion, etc., such as disclosed in the '669 application; atrial fibrillation detection using a distribution of ventricular depolarization intervals, such as disclosed in the '440 application; atrial fibrillation detection using atrial activity scores from an atrial detection window prior to a detected ventricular polarization, such as disclosed in the '565 application; atrial fibrillation discrimination using clustered depolarization information, such as disclosed in the '953 application, etc.

Existing ambulatory medical devices, such as short- or long-term insertable cardiac monitors (ICMs), etc., measure atrial fibrillation burden over a period of time, for example, by determining indications the occurrence of one or both of atrial fibrillation or non-atrial fibrillation in various detection windows, such as each two-minute detection window throughout a day. Atrial fibrillation episodes and physiologic information associated with such, e.g., electrocardiogram (ECG) information, etc., are reported to a clinician. The number of atrial fibrillation episodes and physiologic information associated recorded, transmitted, and reviewed by a clinician are often merely a subset of the total atrial fibrillation burden reported to the clinician or available for review (e.g., stored and not transmitted, transmitted and not reviewed, etc.). In many instances, more burden is reported than is reviewable (e.g., not recorded, stored, or transmitted, etc.).

Certain ambulatory medical devices have limitations, such as based on hardware limitations (e.g., power, processing resources, circuit components, etc.), on how many episodes can be recorded in a period of time (e.g., 5 detected episodes per day, etc.), how much physiologic information can be recorded in a single episode (e.g., 6 minutes per episode at a specific sampling frequency, etc.), or how much physiologic information can be transmitted in a period of time (e.g., 30 minutes per day at a specific sampling frequency, etc.), etc. Recorded and transmitted episodes can be reviewed and adjudicated (e.g., by a clinician, using one or more additional processes, or combinations thereof). However, incomplete physiologic information recorded and transmitted by such ambulatory medical devices having limitations (e.g., having ECG information from a portion of the determined atrial fibrillation episode and a length of the determined atrial fibrillation episode (e.g., the number of detection windows) but not representative information from each detection window of the determined atrial fibrillation episode except that such detection window was determined to be an atrial fibrillation episode) may result in a greater number of false positive adjudications than actually occurred. Moreover, devices and algorithms can learn from adjudications, such that false positive adjudications can directly be fed back to ambulatory medical devices to conserve processing, storage, and transmission resources, further reducing the likelihood that false positive episodes will consume any limited resources, or reducing resources for operation, extending the lifespan or usable life of the ambulatory medical device. In other examples, clinicians may only be interested in longer segments of atrial fibrillation, such as sustained segments of atrial fibrillation greater than a threshold time (e.g., 10 minutes, etc.), which may not be available by certain limited ambulatory medical devices.

The present inventors have recognized, among other things, that key metrics of atrial fibrillation can be determined and recorded over a period of time (e.g., a day, a portion of a day, or a time period commensurate with a day or portion of a day, etc.) and transmitted for review, such as by a clinician, one or more additional processes, or combinations thereof, to facilitate manual and automatic adjustment of atrial fibrillation burden without substantially impacting existing hardware limitations, enabling information from each detection window having one or more indications of atrial fibrillation, or in certain examples each detection window throughout a day or one or more other time periods, to contribute to atrial fibrillation burden determination in a reviewable manner, such as by a clinician or one or more other processes, without impacting existing hardware limitations. Key metrics of atrial fibrillation can include, for example, information representative of one or more detection windows (e.g., values, morphologies, etc.), requiring less storage and transmission resources than recorded ECG information. In an example, key metrics of atrial fibrillation can include physiologic information recorded (and in certain examples detected) at a second lower resolution than ECG information recorded at the first resolution. In certain examples, key metrics of atrial fibrillation can be determined and compared to commensurate metrics for adjudicated episodes of atrial fibrillation (e.g., with associated recorded physiologic information, such as recorded ECG information, etc.). In an example, atrial fibrillation burden for the patient can be determined based on a comparison of the recorded key metrics and commensurate metrics for adjudicated episodes. For example, if the recorded key metrics are similar to commensurate metrics for adjudicated episodes, such as within a threshold amount (e.g., a percentage, etc.), then atrial fibrillation burden for the patient can be determined with respect to the determined burden of the adjudicated episode. In other examples, atrial fibrillation burden for the patient can be determined based on a measure of dissimilarity between the recorded key metrics and commensurate metrics for adjudicated episodes. Some information from one or more detection windows, or each detection window, for review and adjudication provides an improvement in detecting false positive episodes and improves confidence of determinations, such as in contrast to adjudicated episodes having the benefit of recorded ECG information, etc.

In certain examples, a comparison of the recorded key metrics for the patient and commensurate metrics for one or more adjudicated episodes can be presented to a clinician or automated algorithm for review to adjust the atrial fibrillation burden. In certain examples, an automated algorithm, such as performed by one or more processors or circuits, such as an assessment circuit, etc., can determine a most similar or most dissimilar adjudicated episode for presentation to the clinician for review, such as based on the recorded key metrics for the patient. Depending on the results of the comparison (e.g., within or outside of a threshold similarity), the atrial fibrillation burden can be increased (e.g., as a function of the comparison) or not increased.

Key metrics can include, among other things, one or more of: detected R-R variability (e.g., R-R variability exceeding a first AF variability threshold); not-detected R-R variability (e.g., R-R variability not exceeding the first AF variability threshold, or not exceeding one or more other thresholds); heart rate (e.g., average heart rate over a detection window, variability or variance of heart rate over the detection window, or one or more other heart rate metrics); measures of R-R variability; measures of timing intervals or variation in timing intervals or rate changes between successive valid R waves; measures of R-wave morphology (e.g., a percentage of beats in a detection window having a correspondence within a threshold of a patient-specific or population-specific normal sinus rhythm (NSR) template, within a threshold of the preceding beat, etc.); measures of a P-wave or an indication of a presence (or alternatively an absence) of a P-wave or P-waves in a beat or a percentage of beats in a detection window; or one or more other measures used by an atrial fibrillation algorithm to determine atrial fibrillation for a patient, such as described by or disclosed in one or more of the applications incorporated by reference herein. For example, key metrics can include measures of successive beats or groups of beats having detected R-R variability exceeding a threshold, or separately, not exceeding a threshold (e.g., little or no detected R-R variability, etc.), such as moving averages over groups of beats throughout a day, etc. In certain examples, a value of atrial fibrillation burden can accumulate throughout a day as a function of one or more key metrics, and an atrial fibrillation alert can be determined and provided to a clinician or process as the value of atrial fibrillation burden exceeds a threshold value. In certain examples, the threshold value can be determined as a patient-specific value, such as based on one or more determinations by a clinician or process with respect to the patient or previous information from the patient, or a population value based on one or more clinical determinations with respect to one or more other patients.

In certain examples, key metrics can be determined and recorded for periods or detection windows having one or more indications of atrial fibrillation. In other examples, key metrics can be determined and recorded for each period or detection window, such as throughout a day, such as to allow for comparison of determined periods or detection windows of atrial fibrillation against periods or detection windows not having indications of atrial fibrillation.

In other examples, key metrics can be determined and recorded for each detection window of an atrial fibrillation episode, including for detection windows where ECG information cannot be recorded, such as due to hardware limitations (e.g., limited ambulatory medical devices described above, etc.). In such examples, comparisons of information for each detection window in an episode can be determined in direct contrast to existing ambulatory medical devices having hardware limitations restricting recording or transmitting information about each detection window in a determined atrial fibrillation episode, more than only that each detection window in the determined atrial fibrillation episode was determined to be part of an atrial fibrillation episode.

In other examples, information about one or more interventions or therapies can be recorded and key metrics can be adjusted, tagged, or otherwise noted and comparisons to adjudicated episodes can be adjusted based on the recorded intervention or therapy. For example, information about beta-blocker usage, which can reduce natural heart rate, can be received and recorded. In response, determination of atrial fibrillation burden can be adjusted, such as reducing a weight of heart-rate dependent measures of atrial fibrillation (or increasing other measures) in one or more functions to determine atrial fibrillation burden. Other interventions or therapies include ablation, pacing therapy, antiarrhythmic medications (e.g., class I, II, III, IV, etc.), etc. In other examples, determination of most similar or most dissimilar adjudicated episodes for comparison or presentation can be selected with respect to the specific recorded intervention or therapy.

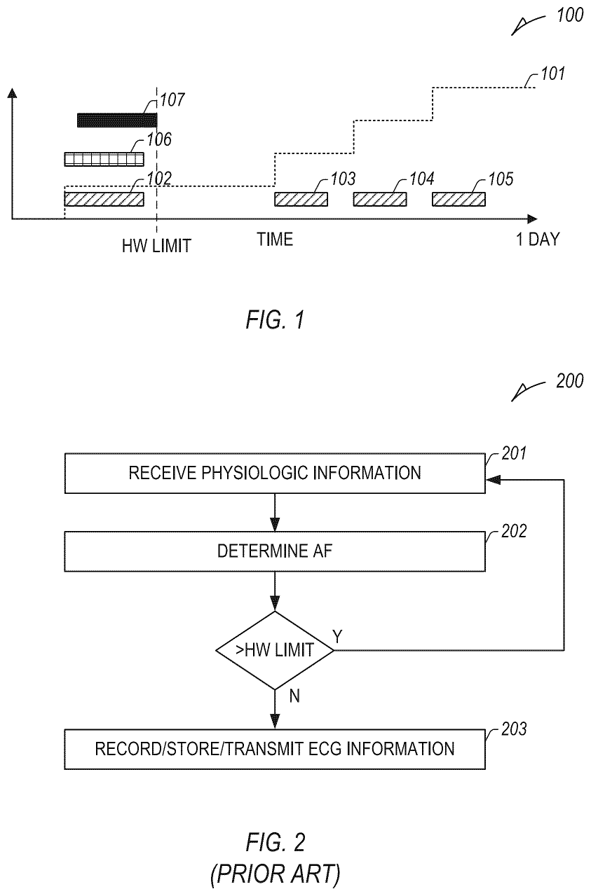

FIG. 1 illustrates an example diagram 100 of daily atrial fibrillation burden 101 for a patient across a period of time (e.g., a day) including detected indications of atrial fibrillation burden 102-105 throughout the period of time, and a detected atrial fibrillation episode 106 and a recorded period of physiologic information 107 (e.g., recorded ECG information) occurring prior to a hardware limitation (HW limit) (e.g., one or more hardware limitations on episodes, detection windows, time periods, or physiologic information that can be recorded or transmitted over the period of time (e.g., a day), etc.).

For example, common sampling rates for ECG information are 250 to 1000 samples per second. For a 6-minute duration (360 seconds) with a sampling rate of 500 samples per second, 180,000 samples can be required. At 2 bytes per sample, 360 kilobytes of storage can be required to store the 6-minute ECG signal. Hardware limitations, in certain examples, can include storage limitations that once exceeded, limits information available for review of confirmation for subsequent events or determinations of atrial fibrillation burden. In other examples, other sampling frequencies can be used, for example, below 250 samples per second, such as greater than 100 samples per second, 200 samples per second, etc., or in certain examples above 1000 samples per second.

Certain ambulatory medical devices can continue to determine and aggregate atrial fibrillation burden throughout the day, even after exceeding one or more hardware limitations. However, such determinations after hardware limitations are often not able to be validated due to such hardware limitations.

FIG. 2 illustrates an example prior art method 200 for determining an indication of atrial fibrillation and recording ECG information associated with such determined indication of atrial fibrillation with existing hardware limitations. At step 201, physiologic information, such as cardiac electrical information (e.g., heart rate, R-R variability, rate or interval information occurring over successive or groups of valid beats, etc.), can be received from a patient over one or more detection windows occurring over a period of time (e.g., a day), for example, using a signal receiver circuit. At step 202, one or more indications of atrial fibrillation (AF) or atrial fibrillation burden can be determined using the received physiologic information, for example, using an assessment circuit. If one or more hardware limitations, such as for the period of time (e.g., a day), are not exceeded, such as by one or more implantable or ambulatory medical devices, for example, detecting physiologic information from the patient, at step 203, ECG information of the patient associated with the detected indication of atrial fibrillation or atrial fibrillation burden can be recorded, stored, or transmitted by the implantable or ambulatory medical device, such as using the assessment circuit. However, if one or more hardware limitations are exceeded, additional physiologic information, such as ECG information of the patient, is not recorded, resulting in additional atrial fibrillation burden determinations not having recorded information to verify or review such determinations.

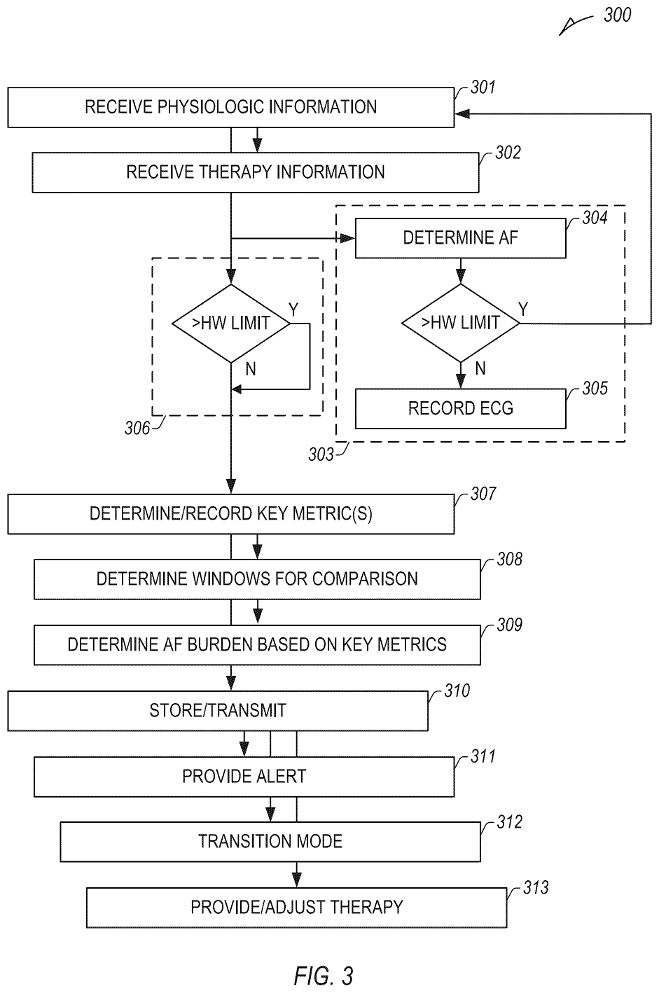

FIG. 3 illustrates an example method 300 for determining and recording one or more key metrics of atrial fibrillation or atrial fibrillation burden over a period of time (e.g., a day, a portion of a day, or a time period commensurate with a day or portion of a day, etc.) without substantially exceeding or impacting existing hardware limitations, enabling information from each detection window having one or more indications of atrial fibrillation, or in certain examples each detection window throughout a day or one or more other time periods, to contribute to atrial fibrillation burden determination in a reviewable manner, such as by a clinician or one or more other processes, without substantially exceeding or impacting existing hardware limitations. In contrast to recorded ECG information, often requiring hundreds of kilobytes of storage and transmission, key metrics of atrial fibrillation or atrial fibrillation burden can often be several bytes, tens of bytes, etc., substantially less than the recorded ECG information.

For example, whereas recorded ECG information can require storage of 500 samples per second, such as described above, recorded key metrics of atrial fibrillation burden, such as representative information described above and herein, can require merely one or several samples (e.g., for each type of representative information) per detection window, per several detection windows, or per episode. For one or two recorded key metrics for a 2-minute detection window, instead of 500 samples per second (for recorded ECG information), recorded key metrics may require as little as one or two (e.g., in certain examples less than 5, less than 10, etc.) samples per a detection window (in this example 120 seconds), substantially less information than recorded ECG information (e.g., a factor of 30,000-60,000 less), the addition of which would not substantively exceed or impact existing hardware limitations. In such case, even covering each 2-minute detection window throughout the day, whether having a determined indication of atrial fibrillation or not, would require as little as 720 samples for the entire day, less than 2 seconds of additional recorded ECG information at 500 samples per second (e.g., in contrast to existing hardware limitations allotting for at least several minutes of such information). To account for such additional samples, ambulatory medical devices can be afforded the additional samples in hardware limitations, or in certain examples existing limitations can be reduced by a commensurate amount to account for such additional samples (e.g., reducing an existing 6-minute recorded ECG allotment per day by 1 second, 1.5 seconds, etc.).

At step 301, physiologic information, such as cardiac electrical information (e.g., heart rate, R-R variability, rate or interval information occurring over successive or groups of valid beats, etc.), can be received from a patient over one or more detection windows occurring over a period of time (e.g., a day), for example, using a signal receiver circuit. At step 302, therapy information can be optionally received, for example, including one or more indications of intervention or therapy, such as electrical therapy (e.g., pacing) by one or more medical devices, application or usage of one or more drug or other therapies (e.g., beta blockers, antiarrhythmic medications, ablation, etc.), for example, using the signal receiver circuit.

In certain examples, at step 303, one or more steps from the prior art method 200 illustrated in FIG. 2 can be performed. For example, at step 304, one or more indications of atrial fibrillation (AF) or atrial fibrillation burden can be determined using the received physiologic information, such as using an assessment circuit. If one or more hardware limitations of one or more implantable or ambulatory medical devices or components thereof, such as for the period of time (e.g., a day), are not exceeded, for example, detecting physiologic information from the patient, then at step 305, ECG information of the patient associated with the detected indication of atrial fibrillation or atrial fibrillation burden can be recorded, stored, or transmitted by the implantable or ambulatory medical device, such as using an assessment circuit or one or more other components of the implantable or ambulatory medical device, etc.

At step 306, in contrast to step 303 and the prior art method 200 illustrated in FIG. 2, regardless of certain prior art hardware limitations being exceeded or not exceeded, one or more key metrics of atrial fibrillation can be determined and recorded at step 307 for each detection window of an atrial fibrillation episode, including for detection windows where ECG information cannot be recorded, such as due to one or more hardware limitations being exceeded, for example, using the assessment circuit. In certain examples, one or more key metrics of atrial fibrillation can be determined and recorded for each detection window having an indication of atrial fibrillation, such as determined at step 304. In other examples, one or more key metrics of atrial fibrillation can be determined and recorded, such as instead of recording ECG information at step 305, after one or more hardware limitations have been exceeded. In certain examples, one or more key metrics of atrial fibrillation can be determined and recorded for groups of detection windows (e.g., groups of two or more detection windows, all detection windows of respective detected indications of atrial fibrillation, etc.) or one or more key metrics can be determined for recorded episodes of atrial fibrillation spanning one or more detection windows, etc. In certain examples, one or more key metrics can be determined and recorded for each detection window of a determined indication of atrial fibrillation and at least one detection window preceding, following, or proceeding and following the determined indications of atrial fibrillation, such as to provide contextual information around the determined indications of atrial fibrillation or to provide information about a difference between the one or more key metrics leading up to or following the detected indication and the one or more key metrics of the detected indication of atrial fibrillation, such as to aid selection of adjudicated episodes for comparison, to provide additional information to a clinician for review, to more accurately determine an indication of atrial fibrillation burden, etc.

At step 308, one or more detection windows of commensurate metrics for adjudicated episodes can be optionally determined for comparison to recorded key metrics of atrial fibrillation for one or more detection windows, such as to determine atrial fibrillation burden for the one or more detection windows based on the previously adjudicated episodes, reducing clinician burden, increasing the sensitivity and specificity of determinations of atrial fibrillation burden for the patient, and reducing the likelihood of false positive determinations of atrial fibrillation and atrial fibrillation burden for the patient, for example, using the assessment circuit.

At step 309, one or more measures of atrial fibrillation burden can optionally be determined based on the one or more recorded key metrics of atrial fibrillation for the patient, for example, using the assessment circuit. In contrast to the prior art, recorded key metrics of atrial fibrillation determined at step 307 can be used in addition to the determination of atrial fibrillation at step 304, for example, as an additional filtering step or to more accurately determine measures (e.g., one or more values, etc.) of atrial fibrillation burden for the patient or confidence of such determinations as a function of the determined atrial fibrillation burden and an output of a comparison of the recorded key metrics to one or more previously adjudicated episodes, etc., reducing false positives and enabling additional information to be displayed to a clinician or presented to one or more additional processes for adjudication, etc.

At step 310, recorded key metrics of atrial fibrillation can be stored, such as using the assessment circuit, and transmitted, by control of the assessment circuit or using one or more communication circuits, etc., such as to one or more additional processes or components, such as an output circuit (e.g., a display, a controller for a display, etc.). At step 311, an alert can be optionally provided, such as by the assessment circuit, for example, if the recorded key metrics of atrial fibrillation are available for review or transmission, if one or more detection windows for comparison have been determined for adjudication, if one or more comparisons to one or more previously adjudicated episodes have been determined, if one or more of the recorded key metrics of atrial fibrillation exceeds a threshold, or if a difference between recorded key metrics of the same or different detected episode of atrial fibrillation exceeds a threshold or expected value, etc. In an example, an output can be provided of the recorded key metric of atrial fibrillation to a user interface for display to a user or to another circuit to control or adjust a process or a function of an implantable or ambulatory medical device.

At step 312, one or more modes or functions of the assessment circuit or an implantable or ambulatory medical device can be optionally adjusted based on one or more of the recorded key metrics of atrial fibrillation, the determined atrial fibrillation burden, or one or more other measures, values, or metrics. For example, if one or more measures of recorded key metrics of atrial fibrillation or the determined atrial fibrillation burden indicate an expected patient end of life (EOL) longer than the determined estimated remaining battery status of implantable or ambulatory medical device, one or more modes or functions of the implantable or ambulatory medical device can be altered to increase the remaining battery status of the medical device. If the recorded key metrics of atrial fibrillation or the determined atrial fibrillation burden indicate that the expected patient EOL is shorter than the determined estimated remaining battery status of the implantable or ambulatory medical device, one or more modes or functions of the implantable or ambulatory medical device can be altered to improve data collection or sensing or to otherwise provide more patient benefit, reducing the remaining battery status of the medical device, but not shorter than the expected patient EOL, in certain examples, with additional tolerance. For example, one or more hardware limitations can be adjusted, such as to, among others: record more or less ECG information of the patient; increase communication frequency between the implantable or ambulatory medical device and an external device (e.g., remote device, programmer, etc.), such as to increase the frequency of patient monitoring, etc.; switch to a different or more power or resource intensive monitoring algorithm; etc.

At step 313, one or more therapies can be optionally provided or adjusted based on the recorded key metrics of atrial fibrillation, the determined atrial fibrillation burden, or one or more other measures, values, or metrics, such as described herein.

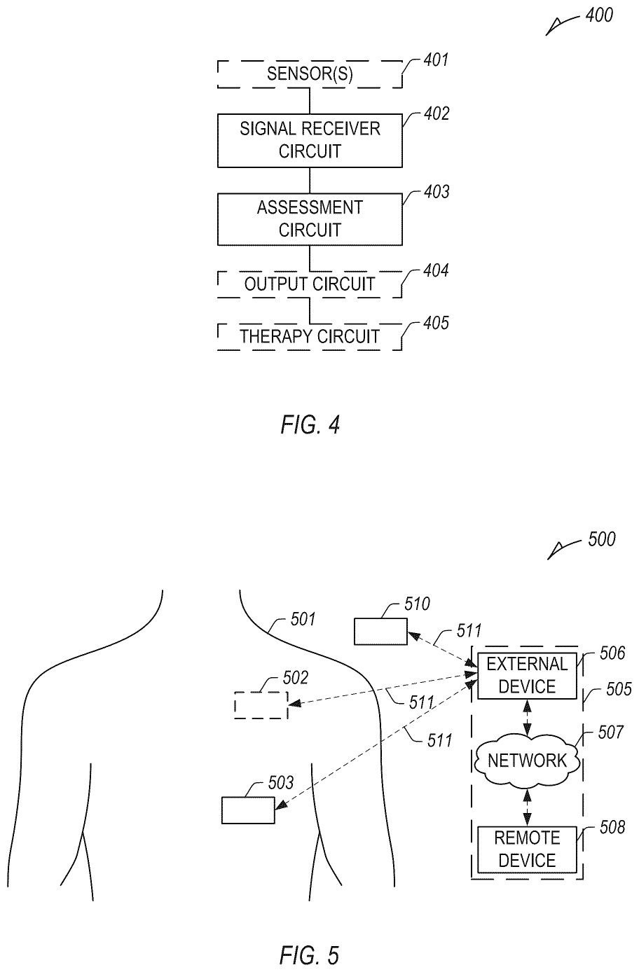

FIG. 4 illustrates an example system 400 (e.g., a medical device system). In an example, one or more aspects of the example system 400 can be a component of, or communicatively coupled to, a medical device, such as an implantable medical device (IMD), an insertable cardiac monitor (ICM), an ambulatory medical device (AMD), etc. The system 400 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 400 can include a single medical device or a plurality of medical devices implanted in a patient's body or otherwise positioned on or about the patient to monitor patient physiologic information of the patient using information from one or more sensors, such as a sensor 401. In an example, the sensor 401 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 400 can include a signal receiver circuit 402 and an assessment circuit 403. The signal receiver circuit 402 can be configured to receive physiologic information of a patient (or group of patients) from the sensor 401. The assessment circuit 403 can be configured to receive information from the signal receiver circuit 402, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 400 can include an output circuit 404 configured to provide an output to a user, or to cause an output to be provided to a user, such as through an output, a display, or one or more other user interface, the output including a score, a trend, an alert, or other indication. In other examples, the output circuit 404 can be configured to provide an output to another circuit, machine, or process, such as a therapy circuit 405 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 405 can include one or more of a stimulation control circuit, a cardiac stimulation circuit, a neural stimulation circuit, a dosage determination or control circuit, etc. In other examples, the therapy circuit 405 can be controlled by the assessment circuit 403, or one or more other circuits, etc. In certain examples, the assessment circuit 403 can include the output circuit 404 or can be configured to determine the output to be provided by the output circuit 404, while the output circuit 404 can provide the signals that cause the user interface to provide the output to the user based on the output determined by the assessment circuit 403.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

FIG. 5 illustrates an example patient management system 500 and portions of an environment in which the patient management system 500 may operate. The patient management system 500 can perform a range of activities, including remote patient monitoring and diagnosis of a disease condition. Such activities can be performed proximal to a patient 501, such as in a patient home or office, through a centralized server, such as in a hospital, clinic, or physician office, or through a remote workstation, such as a secure wireless mobile computing device.

The patient management system 500 can include one or more medical devices, an external system 505, and a communication link 511 providing for communication between the one or more ambulatory medical devices and the external system 505. The one or more medical devices can include an ambulatory medical device (AMD), such as an implantable medical device (IMD) 502, a wearable medical device 503, or one or more other implantable, leadless, subcutaneous, external, wearable, or medical devices configured to monitor, sense, or detect information from, determine physiologic information about, or provide one or more therapies to treat various conditions of the patient 501, such as one or more cardiac or non-cardiac conditions (e.g., dehydration, sleep disordered breathing, etc.).

In an example, the implantable medical device 502 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 501. In another example, the implantable medical device 502 can include a monitor implanted, for example, subcutaneously in the chest of patient 501, the implantable medical device 502 including a housing containing circuitry and, in certain examples, one or more sensors, such as a temperature sensor, etc.

Cardiac rhythm management devices, such as insertable cardiac monitors (ICMs), pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 502 can include an assessment circuit configured to detect or determine specific physiologic information of the patient 501, or to determine one or more conditions or provide information or an alert to a user, such as the patient 501 (e.g., a patient), a clinician, or one or more other caregivers or processes, such as described herein. The implantable medical device 502 can alternatively or additionally be configured as a therapeutic device configured to treat one or more medical conditions of the patient 501. The therapy can be delivered to the patient 501 via the lead system and associated electrodes or using one or more other delivery mechanisms. The therapy can include delivery of one or more drugs to the patient 501, such as using the implantable medical device 502 or one or more of the other ambulatory medical devices, etc. In some examples, therapy can include CRT for rectifying dyssynchrony and improving cardiac function in heart failure patients. In other examples, the implantable medical device 502 can include a drug delivery system, such as a drug infusion pump to deliver drugs to the patient for managing arrhythmias or complications from arrhythmias, hypertension, hypotension, or one or more other physiologic conditions. In other examples, the implantable medical device 502 can include one or more electrodes configured to stimulate the nervous system of the patient or to provide stimulation to the muscles of the patient airway, etc.

The wearable medical device 503 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 505 can include a dedicated hardware/software system, such as a programmer, a remote server-based patient management system, or alternatively a system defined predominantly by software running on a standard personal computer. The external system 505 can manage the patient 501 through the implantable medical device 502 or one or more other ambulatory medical devices connected to the external system 505 via a communication link 511. In other examples, the implantable medical device 502 can be connected to the wearable medical device 503, or the wearable medical device 503 can be connected to the external system 505, via the communication link 511. This can include, for example, programming the implantable medical device 502 to perform one or more of acquiring physiologic data, performing at least one self-diagnostic test (such as for a device operational status), analyzing the physiologic data, or optionally delivering or adjusting a therapy for the patient 501. Additionally, the external system 505 can send information to, or receive information from, the implantable medical device 502 or the wearable medical device 503 via the communication link 511. Examples of the information can include real-time or stored physiologic data from the patient 501, diagnostic data, such as detection of patient hydration status, hospitalizations, responses to therapies delivered to the patient 501, or device operational status of the implantable medical device 502 or the wearable medical device 503 (e.g., battery status, lead impedance, etc.). The communication link 511 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 505 can include an external device 506 in proximity of the one or more ambulatory medical devices, and a remote device 508 in a location relatively distant from the one or more ambulatory medical devices, in communication with the external device 506 via a communication network 507. Examples of the external device 506 can include a medical device programmer. The remote device 508 can be configured to evaluate collected patient or patient information and provide alert notifications, among other possible functions. In an example, the remote device 508 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 508 can receive data from multiple patients. The data can be collected by the one or more ambulatory medical devices, among other data acquisition sensors or devices associated with the patient 501. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 508 may additionally include one or more locally configured clients or remote clients securely connected over the communication network 507 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 508, including the server and the interconnected clients, may also execute a follow-up scheme by sending follow-up requests to the one or more ambulatory medical devices, or by sending a message or other communication to the patient 501 (e.g., the patient), clinician or authorized third party as a compliance notification.

The communication network 507 can provide wired or wireless interconnectivity. In an example, the communication network 507 can be based on the Transmission Control Protocol/Internet Protocol (TCP/IP) network communication specification, although other types or combinations of networking implementations are possible. Similarly, other network topologies and arrangements are possible.

One or more of the external device 506 or the remote device 508 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 506 or the remote device 508 can include a respective display unit for displaying the physiologic or functional signals, or alerts, alarms, emergency calls, or other forms of warnings to signal the detection of arrhythmias. In some examples, the external system 505 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 505 can be implemented using hardware, software, firmware, or combinations thereof. Portions of the one or more ambulatory medical devices or the external system 505 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 510 can be configured to send information to or receive information from one or more of the ambulatory medical devices or the external system 505 using the communication link 511. In an example, the one or more ambulatory medical devices, the external device 506, or the remote device 508 can be configured to control one or more parameters of the therapy device 510. The external system 505 can allow for programming the one or more ambulatory medical devices and can receives information about one or more signals acquired by the one or more ambulatory medical devices, such as can be received via a communication link 511. The external system 505 can include a local external implantable medical device programmer. The external system 505 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

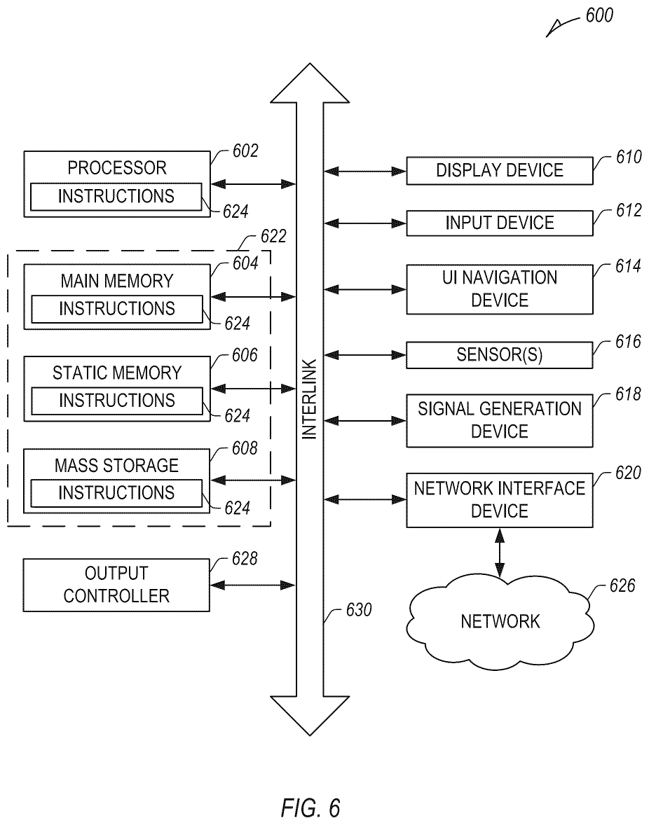

FIG. 6 illustrates a block diagram of an example machine 600 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 600. Circuitry (e.g., processing circuitry, an assessment circuit, etc.) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 600 (e.g., computer system) may include a hardware processor 602 (e.g., a central processing unit (CPU), a graphics processing unit (GPU), a hardware processor core, or any combination thereof), a main memory 604, a static memory 606 (e.g., memory or storage for firmware, microcode, a basic-input-output (BIOS), unified extensible firmware interface (UEFI), etc.), and mass storage 608 (e.g., hard drive, tape drive, flash storage, or other block devices) some or all of which may communicate with each other via an interlink 630 (e.g., bus). The machine 600 may further include a display unit 610, an input device 612 (e.g., a keyboard), and a user interface (UI) navigation device 614 (e.g., a mouse). In an example, the display unit 610, input device 612, and UI navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 602, the main memory 604, the static memory 606, or the mass storage 608 may be, or include, a machine-readable medium 622 on which is stored one or more sets of data structures or instructions 624 (e.g., software) embodying or utilized by any one or more of the techniques or functions described herein. The instructions 624 may also reside, completely or at least partially, within any of registers of the hardware processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive physiologic information of a patient; and
an assessment circuit configured to:
determine indications of atrial fibrillation of the patient in respective detection windows of a day using the received physiologic information;
record first physiologic information of the patient at a first sampling frequency for the determined indications of atrial fibrillation of the patient up to and not exceeding a first threshold of the medical device system for transmission to a remote device; and
determine and record one or more key metrics of atrial fibrillation for the determined indications of atrial fibrillation of the patient at a second sampling frequency lower than the first sampling frequency without regard to the first threshold.

2. The medical device system of claim 1, wherein to determine and record the one or more key metrics of atrial fibrillation for the determined indications of atrial fibrillation of the patient without regard to the first threshold comprises to determine and record one or more key metrics of atrial fibrillation for the determined indications of atrial fibrillation without regard to a limit, including to determine and record one or more key metrics of atrial fibrillation for the determined indications of atrial fibrillation up to and exceeding the first threshold.

3. The medical device system of claim 1, wherein the first threshold is representative of a first data limit for storage or transmission by the assessment circuit for a determined indication of atrial fibrillation, a time period, or a period between sequential transmissions to the remote device.

4. The medical device system of claim 1, wherein the first threshold comprises a daily threshold for determined indications of atrial fibrillation,
wherein the assessment circuit is configured to cease recording the first physiologic information of the patient for a remainder of the day after an amount of the recorded first physiologic information for the day has met or exceeded the first threshold.

5. The medical device system of claim 1, wherein the assessment circuit is configured to determine and record the one or more key metrics of atrial fibrillation for one or more detection windows of each of the determined indications of atrial fibrillation of the patient for transmission to the remote device,
wherein each determined indication of atrial fibrillation comprises one or more detection windows.

6. The medical device system of claim 1, wherein the assessment circuit is configured to determine and record the one or more key metrics of atrial fibrillation for each detection window of the determined indications of atrial fibrillation,
wherein each determined indication of atrial fibrillation comprises one or more detection windows.

7. The medical device system of claim 1, wherein the assessment circuit is configured to determine and record the one or more key metrics of atrial fibrillation for each detection window of the determined indications of atrial fibrillation and at least one detection window preceding or following the determined indications of atrial fibrillation,
wherein each determined indication of atrial fibrillation comprises one or more detection windows.

8. The medical device system of claim 1, wherein the assessment circuit is configured to determine and record the one or more key metrics of atrial fibrillation for each detection window of the day, including detection windows with and without determined indications of atrial fibrillation.

9. The medical device system of claim 1, comprising an implantable or an ambulatory medical device including the signal receiver circuit and the assessment circuit,
wherein the detection window has a duration between 30 seconds and 2 minutes,
wherein the first physiologic information of the patient at the first sampling frequency comprises an ECG signal of the patient at a sampling frequency greater than 100 samples per second for the determine indications of atrial fibrillation up to and not exceeding the first threshold of the medical device system,
wherein the one or more key metrics of atrial fibrillation comprise one or more measures or values representative of one or more detection windows of the determined indications at the second sampling frequency having fewer than 5 samples per detection window.

10. The medical device system of claim 1, wherein each determined indication of atrial fibrillation comprises one or more detection windows,
wherein the one or more key metrics of atrial fibrillation comprise, for the one or more detection windows of each determined indication of atrial fibrillation, a measure or value of one or more of:
R-R variability;
timing intervals between successive valid R waves;
R-wave morphology;
P-wave presence; and
heart rate.

11. The medical device system of claim 10, wherein the measure or value of one or more of R-R variability, timing intervals between successive valid R waves, and heart rate comprises an indication that one or more of R-R variability, timing intervals between successive valid R waves, and heart rate is above or below a patient-specific or population threshold for the one or more detection windows of each determined indication of atrial fibrillation.

12. The medical device system of claim 1, wherein the assessment circuit is configured to determine an indication of atrial fibrillation burden for the patient using the one or more recorded key metrics of atrial fibrillation.

13. The medical device system of claim 12, wherein the assessment circuit is configured to determine the indication of atrial fibrillation burden for the patient using a comparison of the one or more recorded key metrics to commensurate metrics for adjudicated episodes.

14. A method, comprising:
receiving physiologic information of a patient using a signal receiver circuit; and
using an assessment circuit:
determining indications of atrial fibrillation of the patient in respective detection windows of a day using the received physiologic information;
recording first physiologic information of the patient at a first sampling frequency for the determined indications of atrial fibrillation of the patient up to and not exceeding a first threshold for transmission to a remote device; and
determining and recording one or more key metrics of atrial fibrillation for the determined indications of atrial fibrillation of the patient at a second sampling frequency lower than the first sampling frequency without regard to the first threshold.

15. The method of claim 14, wherein determining and recording the one or more key metrics of atrial fibrillation for the determined indications of atrial fibrillation of the patient without regard to the first threshold comprises determining and recording one or more key metrics of atrial fibrillation for the determined indications of atrial fibrillation without regard to a limit, including determining and recording one or more key metrics of atrial fibrillation for the determined indications of atrial fibrillation up to and exceeding the first threshold.

16. The method of claim 14, wherein the first threshold comprises a daily threshold for determined indications of atrial fibrillation,
wherein the method includes, using the assessment circuit, ceasing recording the first physiologic information of the patient for a remainder of the day after an amount of the recorded first physiologic information for the day has met or exceeded the first threshold,
wherein determining and recording the one or more key metrics of atrial fibrillation comprises determining and recording the one or more key metrics of atrial fibrillation for each detection window of the day, including detection windows with and without determined indications of atrial fibrillation.

17. The method of claim 14, wherein determining and recording the one or more key metrics of atrial fibrillation comprises determining and recording the one or more key metrics of atrial fibrillation for one or more detection windows of each of the determined indications of atrial fibrillation of the patient for transmission to the remote device,
wherein each determined indication of atrial fibrillation comprises one or more detection windows.

18. The method of claim 14, wherein the detection window has a duration between 30 seconds and 2 minutes,
wherein the first physiologic information of the patient at the first sampling frequency comprises an ECG signal of the patient at a sampling frequency greater than 100 samples per second for the determine indications of atrial fibrillation up to and not exceeding the first threshold,
wherein the one or more key metrics of atrial fibrillation comprise one or more measures or values representative of one or more detection windows of the determined indications at the second sampling frequency having fewer than 5 samples per detection window.

19. The method of claim 14, wherein each determined indication of atrial fibrillation comprises one or more detection windows,
wherein the one or more key metrics of atrial fibrillation comprise, for the one or more detection windows of each determined indication of atrial fibrillation, a measure or value of one or more of:
R-R variability;
timing intervals between successive valid R waves;
R-wave morphology;
P-wave presence; and
heart rate.

20. The method of claim 14, comprising:
determining an indication of atrial fibrillation burden for the patient using a comparison of the one or more recorded key metrics to commensurate metrics for adjudicated episodes.
